(19) United States
(12) Patent Application Publication
(10) Pub. No.: US 2026/0355753 A1
(43) Pub. Date: Nov. 12, 2026

(54) PARTICLE SORTING DEVICE AND METHOD

(71) Applicant: Hangzhou POWCLIN Medical Technology Co., Ltd., Hangzhou City (CN)

(72) Inventors: Chuanchao ZHANG, Hangzhou (CN); Rui LI, Hangzhou (CN); Xiaofeng YU, Hangzhou (CN); Shuanglai HAN, Hangzhou (CN); Yang LIU, Hangzhou (CN); Chun YANG, Hangzhou (CN); Lingfeng WENG, Hangzhou (CN)

(73) Assignee: Hangzhou POWCLIN Medical Technology Co., Ltd., Hangzhou City (CN)

(21) Appl. No.: 19/664,340

(22) Filed: Apr. 30, 2026

(30) Foreign Application Priority Data
May 6, 2025 (CN) 2025105711864

(51) Int. Cl. B03B 5/62

(52) U.S. Cl. CPC B03B 5/62

(57) ABSTRACT

A particle sorting device and method are disclosed. The particle sorting device includes a microfluidic chip, where a first flow channel is provided inside the microfluidic chip; a plurality of channels of a first group of flow channels uniformly surround the first flow channel, and a first inlet and a first outlet of each flow channel in the first group of flow channels are both in communication with the first flow channel; and a plurality of channels of a second group of flow channels uniformly surround the first flow channel, a second inlet of each flow channel in the second group of flow channels is in communication with the first flow channel, and a third included angle γ between a liquid flow direction at the second inlet and a liquid flow direction in the first flow channel is an acute angle.

### CROSS-REFERENCE TO RELATED APPLICATIONS

The present disclosure claims priority to Chinese Patent Application No. 2025105711864, filed with the China National Intellectual Property Administration on May 6, 2025, and entitled "PARTICLE SORTING DEVICE AND METHOD", which is incorporated herein by reference in its entirety.

### TECHNICAL FIELD

The present disclosure relates to cell analysis, and in particular, to a particle sorting device and method.

### BACKGROUND

Conventional flow cytometers have various problems, such as excessively large instrument size, complex operation, and relatively serious damage to cells caused by a high-speed sheath fluid and an ejection process.

After years of development of microfluidic technology in the field of life science, problems such as cell manipulation and feature detection can already be well solved. Therefore, microfluidics-based flow cytometric analyzers and sorters have emerged. Similar to the structure of conventional flow analysis, microfluidic flow cytometry also includes focusing and optical detection modules. When a sorting function is provided, a sorting module is further included. A main difference between the two technologies is that a microfluidic chip has an approximately planar structure and integrates various pipelines into a single chip, so that analysis of cells is implemented in a closed space. Such an arrangement has many advantages, and the most important advantage is avoidance of contamination. A disposable chip avoids contamination between samples, and a closed system eliminates potential aerosol contamination.

A conventional flow sorting method adopts high-speed droplet generation and high-voltage electrified sorting, where a high-speed flowing sample and a high voltage applied to droplets cause unavoidable damage to cells, reduce cell viability, and seriously affect results of subsequent experiments. A sorting module integrated on a microfluidic chip can directly sort target cells and particles in liquid, without generating droplets, thereby avoiding a high-voltage and high-speed liquid environment.

Currently, some microfluidic sorting products have also appeared. However, existing technical methods still have defects mainly in two aspects.
1. A planar microfluidic sorting chip only implements fluid focusing in a planar dimension, which causes a sample flow to be planar rather than linear. The poor focusing effect is unfavorable for optical detection and affects a CV value of an instrument.
2. Sorting speed is relatively low. In most such products, a sorting force is triggered from a direction perpendicular to a sample flow, and a target is sorted by disturbing fluid. Since a period of time is required to restore stability after fluid is disturbed in the perpendicular direction, sorting time is greatly affected.

### SUMMARY

A particle sorting device includes a microfluidic chip, where a first flow channel allowing sheath fluid and a sample to flow therethrough is provided inside the microfluidic chip, and in the first flow channel, the sheath fluid envelops the sample; and the microfluidic chip further includes:
a first group of flow channels, where a plurality of channels of the first group of flow channels uniformly surround the first flow channel, a first inlet and a first outlet of each flow channel in the first group of flow channels are both in communication with the first flow channel, a first included angle α between a liquid flow direction at the first inlet and a liquid flow direction in the first flow channel is an acute angle, and a second included angle β between a liquid flow direction at the first outlet and the liquid flow direction in the first flow channel is an obtuse angle;
a second group of flow channels, where a plurality of channels of the second group of flow channels uniformly surround the first flow channel, a second inlet of each flow channel in the second group of flow channels is in communication with the first flow channel, and a third included angle γ between a liquid flow direction at the second inlet and the liquid flow direction in the first flow channel is an acute angle; the second inlet is arranged between the first inlet and the first outlet;
a first chamber, where the first chamber is in communication with the first flow channel and is arranged downstream of the first outlet; and
an adjustment unit, where the adjustment unit is configured to adjust a volume of the first chamber.

A particle sorting method implemented based on the foregoing particle sorting device includes the following steps:
A1. in the first flow channel, the sheath fluid envelops the sample and flows forward;
A2. a portion of outer sheath fluid enters the first group of flow channels, and remaining sheath fluid continues to envelop the sample and flows forward;
A3. a portion of the outer sheath fluid enters the second group of flow channels; and
A4. the sheath fluid in the first group of flow channels enters the first flow channel from the first outlet in a reverse direction, wherein when sorting is required, the volume of the first chamber increases, and the sample in the first flow channel sequentially flows through the second inlet and the first outlet, flows forward while carrying the sheath fluid flowing in from the first outlet, and enters the first chamber; and
when sorting is not required, the volume of the first chamber remains unchanged or decreases, the sheath fluid entering the first flow channel from the first outlet flows in the reverse direction and mixes with the sample, and enters the second group of flow channels, and no sample passes through the first outlet.

### BRIEF DESCRIPTION OF DRAWINGS

With reference to the accompanying drawings, the disclosed content of the present disclosure will become more readily understood. Those skilled in the art can readily understand that the accompanying drawings are merely used for illustrating the technical solutions of the present disclosure, and are not intended to limit the protection scope of the present disclosure. In the drawings:

FIG. 1 is a schematic diagram of a structure of a particle sorting device according to the present disclosure;

FIG. 2 is a schematic diagram of a partial structure of a particle sorting device according to the present disclosure; and

FIG. 3 is a schematic diagram of a partial structure of a particle sorting device according to the present disclosure.

Reference numerals: 11. microfluidic chip, 21. fourth group of flow channels, 211. second liquid inlet, 31. sample inlet, 41. first flow channel, 411. first portion, 412. second portion, 413. third portion, 51. first group of flow channels, 511. first inlet, 512. first outlet, 61. second group of flow channels, 611. second inlet, 71. third group of flow channels, 711. second outlet, 712. outer sidewall, 713. inner sidewall, 715. first liquid inlet, 81. first chamber, 82. second flow channel, and 91. storage chamber.

### DETAILED DESCRIPTION OF EMBODIMENTS

Embodiments of the present disclosure are described in detail below, and examples of the embodiments are shown in the accompanying drawings, where the same or similar reference numerals throughout represent the same or similar elements or elements having the same or similar functions. The embodiments described below with reference to the accompanying drawings are exemplary and are intended to be used to explain the present disclosure, but should not be construed as limiting the present disclosure.

In the description of the present disclosure, it should be understood that an orientation or a position relationship indicated by the terms "center", "longitudinal", "lateral", "length", "width", "thickness", "upper", "lower", "front", "rear", "left", "right", "vertical", "horizontal", "top", "bottom", "inside", "outside", "clockwise", "counterclockwise", "axial", "radial", "circumferential", or the like is an orientation or position relationship shown in the accompanying drawings, and is merely intended to facilitate description and simplify description of the present disclosure, but is not intended to indicate or imply that a specified apparatus or element must have a specific orientation, be constructed in a specific orientation, and operate in a specific orientation. Therefore, the present disclosure cannot be construed as a limitation.

In addition, the terms "first" and "second" are merely intended for a purpose of description, and shall not be understood as an indication or implication of relative importance or an implicit indication of a quantity of indicated technical features. Therefore, a feature defined with "first" or "second" may explicitly or implicitly include one or more features. In the description of the present disclosure, "a plurality" means two or more unless specifically defined otherwise.

In the present disclosure, unless otherwise specified and limited, the terms such as "mount", "link", "connect", and "fix" should be understood broadly. For example, the term "connect" may be a fixed connection, may be a detachable connection, or may be integration; may be a mechanical connection or may be an electrical connection; or may be a direct connection, may be an indirect connection implemented by using an intermediate medium, or may be communication inside two elements or an interaction relationship between two elements. Those of ordinary skill in the art may interpret specific meanings of the foregoing terms in the present disclosure according to specific cases.

In the present disclosure, unless otherwise expressly specified and defined, a first feature being "on" or "under" a second feature may indicate that the first feature is in direct contact with the second feature, or that the first feature is in indirect contact with the second feature through an intermediate medium. In addition, the first feature being "above", "on top of", and "over" the second feature may include the first feature being directly above or diagonally above the second feature, or simply indicating that the first feature has a higher horizontal height than the second feature. The first feature being "below", "under", and "beneath" the second feature may include the first feature being directly below or diagonally below the second feature, or simply indicating that the first feature has a lower horizontal height than the second feature.

To solve the defects in the above existing technical solutions, the present disclosure provides a microfluidic particle sorting device and method.

An objective of the present disclosure is achieved by the following technical solutions.

The present disclosure provides a particle sorting device, which includes a microfluidic chip, where a first flow channel allowing sheath fluid and a sample to flow therethrough is provided inside the microfluidic chip, and the microfluidic chip further includes:
a first group of flow channels, where a plurality of channels of the first group of flow channels uniformly surround the first flow channel, a first inlet and a first outlet of each flow channel in the first group of flow channels are both in communication with the first flow channel, a first included angle α between a liquid flow direction at the first inlet and a liquid flow direction in the first flow channel is an acute angle, and a second included angle β between a liquid flow direction at the first outlet and the liquid flow direction in the first flow channel is an obtuse angle;
a second group of flow channels, where a plurality of channels of the second group of flow channels uniformly surround the first flow channel, a second inlet of each flow channel in the second group of flow channels is in communication with the first flow channel, and a third included angle γ between a liquid flow direction at the second inlet and the liquid flow direction in the first flow channel is an acute angle; the second inlet is arranged between the first inlet and the first outlet;
a first chamber, where the first chamber is in communication with the first flow channel and is arranged downstream of the first outlet; and
an adjustment unit, where the adjustment unit is configured to adjust a volume of the first chamber.

In one or more embodiments, the first group of flow channels includes two flow channels, the two flow channels are coplanar with the first flow channel, and an outer edge is heart-shaped; and
the second group of flow channels includes two flow channels, and the two flow channels are coplanar with the first flow channel, forming a flared opening.

In one or more embodiments, the first flow channel includes a first portion arranged upstream of the first inlet, a second portion arranged between the first inlet and the first outlet, and a third portion arranged downstream of the first outlet, where an inner diameter of the first portion is greater than an inner diameter of the second portion, the inner diameter of the second portion is greater than an inner diameter of the third portion, and the third portion is in communication with the first chamber.

In one or more embodiments, the adjustment unit includes:
a deformation module and a film, where the film separates an interior of the first chamber from an exterior of the first chamber, and the deformation module is arranged on an outer side of the first chamber and connected to the film.

In one or more embodiments, γ>α>(β-90°), and 30°≤α≤60°.

In one or more embodiments, the microfluidic chip further includes:
a third group of flow channels, where a plurality of channels of the third group of flow channels are all in communication with a first liquid inlet and uniformly surround the first flow channel, a second outlet of each flow channel in the third group of flow channels is in communication with the first flow channel upstream of the first inlet, and a fourth included angle between a liquid flow direction at the second outlet and a liquid flow direction in the first flow channel is an acute angle;
each of an outer sidewall and an inner sidewall of the second outlet is curved and is tangent to an outer wall of the first flow channel, where a radius of curvature of the outer sidewall is 30 mm to 40 mm, a central angle d is 10 degrees to 30 degrees, a radius of curvature of the inner sidewall is 5 mm to 20 mm, a central angle e is 10 degrees to 30 degrees, and d>e; and
a distance between a center of the central angle d and a center of the central angle e measured between perpendicular projection points on the first flow channel is 5 mm to 10 mm.

The present disclosure provides a particle sorting method implemented based on the foregoing particle sorting device, which includes the following steps:
(A1) in the first flow channel, the sheath fluid envelops the sample and flows forward;
(A2) a portion of outer sheath fluid enters the first group of flow channels, and remaining sheath fluid continues to envelop the sample and flows forward;
(A3) a portion of the outer sheath fluid enters the second group of flow channels; and
(A4) the sheath fluid in the first group of flow channels enters the first flow channel from the first outlet in a reverse direction, wherein
when sorting is required, the volume of the first chamber increases, and the sample in the first flow channel sequentially flows through the second inlet and the first outlet, flows forward while carrying the sheath fluid flowing in from the first outlet, and enters the first chamber; and
when sorting is not required, the volume of the first chamber remains unchanged or decreases, the sheath fluid entering the first flow channel from the first outlet flows in the reverse direction and mixes with the sample, and enters the second group of flow channels, and no sample passes through the first outlet.

In one or more embodiments, in the step (A1), the sheath fluid enters the first flow channel in the following manner:
the sheath fluid enters from the first liquid inlet, then enters the first flow channel from the plurality of channels of the third group of flow channels, envelops the sample, and flows forward;
the plurality of channels of the third group of flow channels uniformly surround the first flow channel, the second outlet of each flow channel in the third group of flow channels is in communication with the first flow channel upstream of the first inlet, and a fourth included angle between a liquid flow direction at the second outlet and a liquid flow direction in the first flow channel is an acute angle;
each of an outer sidewall and an inner sidewall of the second outlet is curved and is tangent to an outer wall of the first flow channel, where a radius of curvature of the outer sidewall is 30 mm to 40 mm, a central angle d is 10 degrees to 30 degrees, a radius of curvature of the inner sidewall is 5 mm to 20 mm, a central angle e is 10 degrees to 30 degrees, and d>e; and
a distance between a center of the central angle d and a center of the central angle e measured between perpendicular projection points on the first flow channel is 5 mm to 10 mm.

In one or more embodiments, the volume of the first chamber is adjusted in the following manner:
the deformation module pushes the film in the first chamber to move back and forth, and the volume of the first chamber switches between increasing and decreasing.

The present disclosure provides a particle sorting device, which includes a microfluidic chip, where a first flow channel allowing sheath fluid and a sample to flow therethrough is provided inside the microfluidic chip, and in the first flow channel, the sheath fluid envelops the sample; and the microfluidic chip further includes:
a first group of flow channels, where a plurality of channels of the first group of flow channels uniformly surround the first flow channel, a first inlet and a first outlet of each flow channel in the first group of flow channels are both in communication with the first flow channel, a first included angle α between a liquid flow direction at the first inlet and a liquid flow direction in the first flow channel is an acute angle, and a second included angle β between a liquid flow direction at the first outlet and the liquid flow direction in the first flow channel is an obtuse angle;
a second group of flow channels, where a plurality of channels of the second group of flow channels uniformly surround the first flow channel, a second inlet of each flow channel in the second group of flow channels is in communication with the first flow channel, and a third included angle γ between a liquid flow direction at the second inlet and the liquid flow direction in the first flow channel is an acute angle; the second inlet is arranged between the first inlet and the first outlet;
a first chamber, where the first chamber is in communication with the first flow channel and is arranged downstream of the first outlet; and
an adjustment unit, where the adjustment unit is configured to adjust a volume of the first chamber.

In one or more embodiments, the first group of flow channels includes two flow channels, the two flow channels are coplanar with the first flow channel, and an outer edge is heart-shaped; and
the second group of flow channels includes two flow channels, and the two flow channels are coplanar with the first flow channel, forming a flared opening.

In one or more embodiments, the first flow channel includes a first portion arranged upstream of the first inlet, a second portion arranged between the first inlet and the first outlet, and a third portion arranged downstream of the first outlet, where an inner diameter of the first portion is greater than an inner diameter of the second portion, the inner diameter of the second portion is greater than an inner diameter of the third portion, and the third portion is in communication with the first chamber.

In one or more embodiments, the adjustment unit includes:
a deformation module and a film, where the film separates an interior of the first chamber from an exterior of the first chamber, and the deformation module is arranged on an outer side of the first chamber and connected to the film.
In one or more embodiments, γ>α>(β-90°), and 30°≤α≤60°.

In one or more embodiments, the microfluidic chip further includes:
a third group of flow channels, where the third group of flow channels includes a first liquid inlet and a plurality of channels, the plurality of channels are all in communication with the first liquid inlet and uniformly surround the first flow channel, a second outlet of each flow channel in the third group of flow channels is in communication with the first flow channel upstream of the first inlet, and a fourth included angle between a liquid flow direction at the second outlet and a liquid flow direction in the first flow channel is an acute angle;
each of an outer sidewall and an inner sidewall of the second outlet is curved and is tangent to an outer wall of the first flow channel, where a radius of curvature of the outer sidewall is 30 mm to 40 mm, a central angle d is 10 degrees to 30 degrees, a radius of curvature of the inner sidewall is 5 mm to 20 mm, a central angle e is 10 degrees to 30 degrees, and d>e; and
a distance between a center of the central angle d and a center of the central angle e measured between perpendicular projection points on the first flow channel is 5 mm to 10 mm; and
a fourth group of flow channels, where the fourth group of flow channels includes a second liquid inlet and an arc-shaped channel perpendicular to a horizontal plane in which the second group of flow channels and the first flow channel are located, the arc-shaped channel uniformly surrounds the first flow channel, and the second liquid inlet is in communication with the arc-shaped channel and the first flow channel.

The present disclosure provides a particle sorting method implemented based on the foregoing particle sorting device, where the particle sorting method includes the following steps:
A1. in the first flow channel, the sheath fluid envelops the sample and flows forward;
A2. a portion of outer sheath fluid enters the first group of flow channels, and remaining sheath fluid continues to envelop the sample and flows forward;
A3. a portion of the outer sheath fluid enters the second group of flow channels; and
A4. the sheath fluid in the first group of flow channels enters the first flow channel from the first outlet in a reverse direction, wherein
when sorting is required, the volume of the first chamber increases, and the sample in the first flow channel sequentially flows through the second inlet and the first outlet, flows forward while carrying the sheath fluid flowing in from the first outlet, and enters the first chamber; and
when sorting is not required, the volume of the first chamber remains unchanged or decreases, the sheath fluid entering the first flow channel from the first outlet flows in the reverse direction and mixes with the sample, and enters the second group of flow channels, and no sample passes through the first outlet.

In one or more embodiments, the microfluidic chip further includes:
a third group of flow channels, where the third group of flow channels includes a first liquid inlet and a plurality of channels, the plurality of channels uniformly surround the first flow channel, a second outlet of each channel in the third group of flow channels is in communication with the first flow channel upstream of the first inlet, and a fourth included angle between a liquid flow direction at the second outlet and a liquid flow direction in the first flow channel is an acute angle;
each of an outer sidewall and an inner sidewall of the second outlet is curved and is tangent to an outer wall of the first flow channel, where a radius of curvature of the outer sidewall is 30 mm to 40 mm, a central angle d is 10 degrees to 30 degrees, a radius of curvature of the inner sidewall is 5 mm to 20 mm, a central angle e is 10 degrees to 30 degrees, and d>e; and
a distance between a center of the central angle d and a center of the central angle e measured between perpendicular projection points on the first flow channel is 5 mm to 10 mm; and
a fourth group of flow channels, where the fourth group of flow channels includes a second liquid inlet and an arc-shaped channel perpendicular to a horizontal plane in which the second group of flow channels and the first flow channel are located, the arc-shaped channel uniformly surrounds the first flow channel, and the second liquid inlet is in communication with the arc-shaped channel and the first flow channel;
in the step A1, the sheath fluid enters the first flow channel in the following manner:
the sheath fluid entering the first flow channel sequentially through the second liquid inlet and the arc-shaped channel surrounds the sample and envelops the sample; and thereafter, the sheath fluid enters from the first liquid inlet, then enters the first flow channel from the plurality of channels of the third group of flow channels, envelops the sample, and flows forward.

In one or more embodiments, the adjustment unit includes a deformation module and a film, where the film separates an interior of the first chamber from an exterior of the first chamber, and the deformation module is arranged on an outer side of the first chamber and connected to the film; and
the volume of the first chamber is adjusted in the following manner:
the deformation module pushes the film in the first chamber to move back and forth, and the volume of the first chamber switches between increasing and decreasing.

Compared with the existing technology, the present disclosure has the following beneficial effects.
1. The microfluidic chip is used as a sorting flow cell, thereby implementing sorting with no cross-contamination, no aerosol, low pressure, and low damage.
2. Sheath fluid is uniformly introduced into an outer edge in the first flow channel from two dimensions at different times, thereby implementing microfluidic three-dimensional fluid focusing, achieving a better fluid focusing effect, and facilitating optical detection.
3. A fluid switching design of sheath fluid diversion and return flow helps implement high-speed sorting, does not additionally introduce a sheath fluid switch, and saves sheath fluid.
4. Sorting is performed in a flow direction of the sample, thereby reducing fluid fluctuation and implementing high-speed sorting.

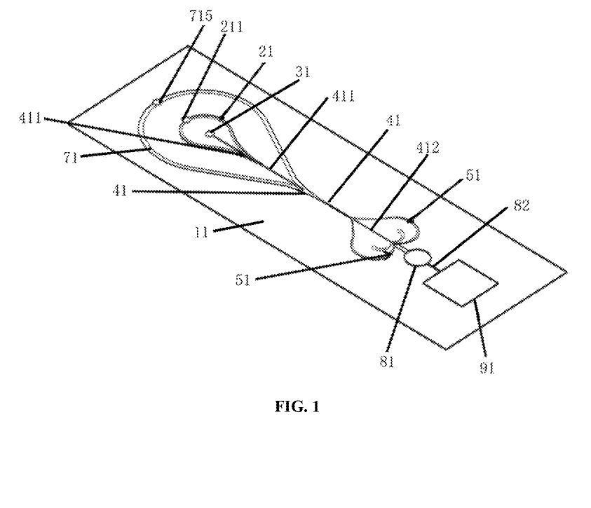

FIGS. 1-3 and the following description describe optional specific embodiments of the present disclosure to teach those skilled in the art how to make and use the present disclosure. To teach the technical solutions of the present disclosure, some conventional aspects have been simplified or omitted. Those skilled in the art should understand that modifications or substitutions derived from these specific embodiments fall within the scope of the present disclosure. Those skilled in the art should understand that the following features can be combined in various ways to form multiple variations of the present disclosure. Therefore, the present disclosure is not limited to the following optional specific embodiments, and is limited only by the claims and equivalents thereof.

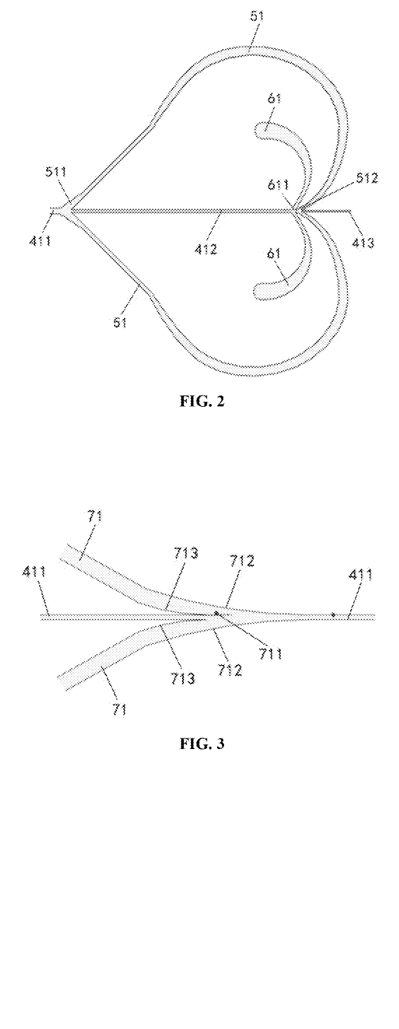

A particle sorting device according to an embodiment of the present disclosure, as shown in FIG. 1, includes a microfluidic chip 11, where the microfluidic chip 11 is internally provided with:
a first flow channel 41 configured to allow sheath fluid and a sample to flow therethrough;
a first group of flow channels 51, as shown in FIG. 2, where a plurality of channels of the first group of flow channels 51 uniformly surround the first flow channel 41, a first inlet 511 and a first outlet 512 of each flow channel in the first group of flow channels 51 are both in communication with the first flow channel 41, a first included angle α between a liquid flow direction at the first inlet 511 and a liquid flow direction in the first flow channel 41 is an acute angle, and a second included angle β between a liquid flow direction at the first outlet 512 and the liquid flow direction in the first flow channel 41 is an obtuse angle;
a second group of flow channels 61, where a plurality of channels of the second group of flow channels 61 uniformly surround the first flow channel 41, a second inlet 611 of each flow channel in the second group of flow channels 61 is in communication with the first flow channel 41, and a third included angle γ between a liquid flow direction at the second inlet 611 and a liquid flow direction in the first flow channel 41 is an acute angle; and the second inlet 611 is arranged between the first inlet 511 and the first outlet 512;
a first chamber 81, where the first chamber 81 is in communication with the first flow channel 41 and is arranged downstream of the first outlet 512; and
an adjustment unit configured to adjust a volume of the first chamber 81, thereby changing pressure in the first chamber 81 and thereby drawing in the sample as required.

To reduce structural complexity, further, the first group of flow channels 51 includes two flow channels, the two flow channels are coplanar with the first flow channel 41, and an outer edge is heart-shaped.

The second group of flow channels 61 includes two flow channels, and the two flow channels are coplanar with the first flow channel 41, forming a flared opening.

To enable sheath fluid at the outer side to envelop an sample at the inner side, further, the first flow channel 41 includes a first portion 411 arranged upstream of the first inlet 511, a second portion 412 arranged between the first inlet 511 and the first outlet 512, and a third portion 413 arranged downstream of the first outlet 512, where an inner diameter of the first portion 411 is greater than an inner diameter of the second portion 412, and the inner diameter of the second portion 412 is greater than an inner diameter of the third portion 413, and the third portion 413 is in communication with the first chamber 81.

To stably adjust the volume of the first chamber 81, further, the adjustment unit includes:
a deformation module and a film, where the film separates an interior of the first chamber 81 from an exterior of the first chamber 81, and the deformation module is arranged on an outer side of the first chamber 81 and connected to the film.

To enable the sheath fluid to stably envelop the sample, further, γ>α>(β-90°), and 30°≤α≤60°.new paragraph...

To better envelop the sample, as shown in FIG. 3, the microfluidic chip 11 further includes:
a third group of flow channels 71, where a plurality of channels of the third group of flow channels 71 are all in communication with a first liquid inlet 715 and uniformly surround the first flow channel 41, a second outlet 711 of each flow channel in the third group of flow channels 71 is in communication with the first flow channel 41 upstream of the first inlet 511, and a fourth included angle between a liquid flow direction at the second outlet 711 and a liquid flow direction in the first flow channel 41 is an acute angle.

Each of an outer sidewall 712 and an inner sidewall 713 of the second outlet 711 is curved and is tangent to an outer wall of the first flow channel 41, where a radius of curvature of the outer sidewall 712 is 30 mm to 40 mm, a central angle d is 10 degrees to 30 degrees, a radius of curvature of the inner sidewall 713 is 5 mm to 20 mm, a central angle e is 10 degrees to 30 degrees, and d>e.

A distance between a center of the central angle d and a center of the central angle e measured between perpendicular projection points on the first flow channel 41 is 5 mm to 10 mm.

A particle sorting method according to an embodiment of the present disclosure, namely a working method of the particle sorting device of the present embodiment, includes the following steps:
(A1) in the first flow channel 41, sheath fluid envelops a sample and flows forward;
(A2) a portion of outer sheath fluid enters the first group of flow channels 51, and remaining sheath fluid continues to envelop the sample and flows forward;
(A3) a portion of the outer sheath fluid enters the second group of flow channels 61; and
(A4) the sheath fluid in the first group of flow channels 51 reversely enters the first flow channel 41 from the first outlet 512, wherein
when sorting is required, the volume of the first chamber 81 increases, and the sample in the first flow channel 41 sequentially flows through the second inlet 611 and the first outlet 512, flows forward while carrying the sheath fluid flowing in from the first outlet 512, and enters the first chamber 81; and
when sorting is not required, the volume of the first chamber 81 remains unchanged or decreases, the sheath fluid entering the first flow channel 41 from the first outlet 512 reversely flows and mixes with the sample, and enters the second group of flow channels 61, and no sample passes through the first outlet 512.

To envelop the sample so as to focus the sample, as shown in FIG. 3, in the step (A1), the sheath fluid enters the first flow channel 41 in the following manner:
the sheath fluid enters from the first liquid inlet 715, then enters the first flow channel 41 from a plurality of channels of the third group of flow channels 71, envelops the sample, and flows forward.

The plurality of channels of the third group of flow channels 71 uniformly surround the first flow channel 41, the second outlet 711 of each flow channel in the third group of flow channels 71 is in communication with the first flow channel 41 upstream of the first inlet 511, and a fourth included angle between a liquid flow direction at the second outlet 711 and a liquid flow direction in the first flow channel 41 is an acute angle.

Each of an outer sidewall 712 and an inner sidewall 713 of the second outlet 711 is curved and is tangent to an outer wall of the first flow channel 41, where a radius of curvature of the outer sidewall 712 is 30 mm to 40 mm, a central angle d is 10 degrees to 30 degrees, a radius of curvature of the inner sidewall 713 is 5 mm to 20 mm, a central angle e is 10 degrees to 30 degrees, and d>e.

A distance between a center of the central angle d and a center of the central angle e measured between perpendicular projection points on the first flow channel 41 is 5 mm to 10 mm.

To stably adjust the volume of the first chamber 81, further, the volume of the first chamber 81 is adjusted in the following manner:
the deformation module pushes a film in the first chamber 81 to move back and forth, and the volume of the first chamber 81 switches between increasing and decreasing.

An application example of the particle sorting device and method according to the foregoing embodiments of the present disclosure in cell sorting is provided.

In this application example, as shown in FIG. 1, the first flow channel 41 includes a first portion 411 (a horizontal width of 75 μm), a second portion 412 (a horizontal width of 50 μm), and a third portion 413 (a horizontal width of 25 μm), which are sequentially arranged and linear. The third portion 413 is sequentially in communication with the first chamber 81, the second flow channel 82, and a storage chamber 91.

As shown in FIG. 2, the first group of flow channels 51 includes two flow channels coplanar with the first flow channel 41 (horizontal plane), and an outer edge is heart-shaped. The two channels symmetrically and uniformly surround the first flow channel 41. The first inlet 511 and the first outlet 512 of each flow channel are both in communication with the first flow channel 41. A first included angle α between a liquid flow direction at the first inlet 511 and a liquid flow direction in the first flow channel 41 is 35 degrees, and a second included angle β between a liquid flow direction at the first outlet 512 and the liquid flow direction in the first flow channel 41 is 120 degrees. The first flow channel 41 between the first inlet 511 and the first outlet 512 is the second portion 412.

The second group of flow channels 61 includes two flow channels coplanar with the first flow channel 41 (horizontal plane) and is ω-shaped. The two channels symmetrically and uniformly surround the second portion 412 of the first flow channel 41. The second inlet 611 of each flow channel in the second group of flow channels 61 is in communication with the first flow channel 41. A third included angle γ between a liquid flow direction at the second inlet 611 and a liquid flow direction in the first flow channel 41 is 60 degrees, satisfying γ>α>(β-90°). The second inlet 611 is arranged between the first inlet 511 and the first outlet 512. An outlet of the second group of flow channels 61 is in communication with a waste liquid reservoir.

As shown in FIG. 3, the third group of flow channels 71 includes a first liquid inlet 715 and two arc-shaped channels horizontally arranged in the microfluidic chip 11, and uniformly surrounds the first flow channel 41. The second outlet 711 of each arc-shaped channel is in communication with the first flow channel 41 upstream of the first inlet 511 (namely, the first portion 411). A fourth included angle between a liquid flow direction at the second outlet 711 and a liquid flow direction in the first flow channel 41 is an acute angle.

Each of an outer sidewall 712 and an inner sidewall 713 of the second outlet 711 is curved and is tangent to an outer wall of the first flow channel 41. A radius of curvature of the outer sidewall 712 is 30 mm to 40 mm, such as 33.4 mm, a central angle d is 10 degrees to 30 degrees, such as 20 degrees, a radius of curvature of the inner sidewall 713 is 5 mm to 20 mm, such as 12.6 mm, a central angle e is 10 degrees to 30 degrees, such as 18 degrees, and d>e.

A distance between a center of the central angle d and a center of the central angle e measured between perpendicular projection points on the first flow channel 41 is 5 mm to 10 mm, such as 6.5 mm.

The fourth group of flow channels 21 includes a second liquid inlet 211 and two arc-shaped channels arranged in the microfluidic chip 11 and perpendicular to the horizontal plane, and symmetrically and uniformly surrounds the first portion 411 of the first flow channel 41.

The sections of the above flow channels perpendicular to central axes thereof are rectangular.

A particle sorting method according to an embodiment of the present disclosure, namely a working method of the particle sorting device of the present embodiment, includes the following steps (A1) to (A4):
(A1) First, a sample enters the first portion 411 through the sample inlet 31.
Then, the sheath fluid sequentially entering the first portion 411 through the second liquid inlet 211 and the fourth group of flow channels 21 surrounds the sample and envelops the sample in a section perpendicular to a central axis of the first portion 411.
Thereafter, the sheath fluid sequentially entering the first portion 411 through the first liquid inlet 715 and the third group of flow channels 71 surrounds the sample and envelops the sample in a section perpendicular to the central axis of the first portion 411, and flows forward in the first flow channel 41
(A2) A portion of outer sheath fluid enters the first group of flow channels 51, and remaining sheath fluid continues to envelop the sample and flows forward in the second portion 412.
(A3) A portion of the outer sheath fluid enters the second group of flow channels 61.
(A4) The sheath fluid in the first group of flow channels 51 reversely enters the first flow channel 41 from the first outlet 512.

When sorting is required, a piezoelectric crystal drives the film to move, so that the volume of the first chamber 81 increases. The sample enveloped by the sheath fluid in the second portion 412 sequentially flows through the second inlet 611 and the first outlet 512, flows forward while carrying the sheath fluid flowing in from the first outlet 512, enters the first chamber 81, and then enters the storage chamber 91 through the second flow channel 82.

When sorting is not required, the volume of the first chamber 81 remains unchanged or decreases, the sheath fluid entering the first flow channel 41 from the first outlet 512 reversely flows and mixes with the sample in the second portion 412, and enters the second group of flow channels 61, and no sample enters the third portion 413.

The differences between an application example of the particle sorting device and method in cell sorting in the foregoing embodiments of the present disclosure and the above embodiments are as follows.
1. The volume of the first chamber 81 is increased.
2. The storage chamber 91 is no longer provided.

### INDUSTRIAL APPLICABILITY

The particle sorting device and method provided by the present disclosure use a microfluidic chip as a sorting flow cell, thereby implementing sorting with no cross-contamination, no aerosol contamination, low pressure, and low damage; sheath fluid is uniformly introduced into an outer edge in the first flow channel from two dimensions at different times, thereby implementing microfluidic three-dimensional fluid focusing, achieving a better fluid focusing effect, and facilitating optical detection; a fluid switching design of sheath fluid diversion and return flow helps implement high-speed sorting, does not additionally introduce a sheath fluid switch, and saves sheath fluid; and the sorting is performed in a flow direction of the sample, thereby reducing fluid fluctuation and implementing high-speed sorting.

1. A particle sorting device, comprising a microfluidic chip, wherein a first flow channel allowing a sheath fluid and a sample to flow therethrough is provided inside the microfluidic chip, and in the first flow channel, the sheath fluid envelops the sample; and the microfluidic chip further comprises:
a first group of flow channels, wherein a plurality of channels of the first group of flow channels uniformly surround the first flow channel, a first inlet and a first outlet of each flow channel in the first group of flow channels are both in communication with the first flow channel, a first included angle α between a liquid flow direction at the first inlet and a liquid flow direction in the first flow channel is an acute angle, and a second included angle β between a liquid flow direction at the first outlet and the liquid flow direction in the first flow channel is an obtuse angle;
a second group of flow channels, wherein a plurality of channels of the second group of flow channels uniformly surround the first flow channel, a second inlet of each flow channel in the second group of flow channels is in communication with the first flow channel, and a third included angle γ between a liquid flow direction at the second inlet and the liquid flow direction in the first flow channel is an acute angle; and the second inlet is arranged between the first inlet and the first outlet;
a first chamber, wherein the first chamber is in communication with the first flow channel and is arranged downstream of the first outlet; and
an adjustment unit, wherein the adjustment unit is configured to adjust a volume of the first chamber.

2. The particle sorting device according to claim 1, wherein the first group of flow channels comprises two flow channels, the two flow channels are coplanar with the first flow channel, and an outer edge is heart-shaped; and
the second group of flow channels comprises two flow channels, and the two flow channels are coplanar with the first flow channel, forming a flared opening.

3. The particle sorting device according to claim 1, wherein the first flow channel comprises a first portion arranged upstream of the first inlet, a second portion arranged between the first inlet and the first outlet, and a third portion arranged downstream of the first outlet, an inner diameter of the first portion is greater than an inner diameter of the second portion, the inner diameter of the second portion is greater than an inner diameter of the third portion, and the third portion is in communication with the first chamber.

4. The particle sorting device according to claim 1, wherein the adjustment unit comprises:
a deformation module and a film, wherein the film separates an interior of the first chamber from an exterior of the first chamber, and the deformation module is arranged on an outer side of the first chamber and connected to the film.

5. The particle sorting device according to claim 1, wherein γ>α>(β-90°), and 30°≤α≤60°.

6. The particle sorting device according to claim 1, wherein the microfluidic chip further comprises:
a third group of flow channels, wherein the third group of flow channels comprise a first liquid inlet and a plurality of channels, the plurality of channels are all in communication with the first liquid inlet and uniformly surround the first flow channel, a second outlet of each flow channel in the third group of flow channels is in communication with the first flow channel upstream of the first inlet, and a fourth included angle between a liquid flow direction at the second outlet and the liquid flow direction in the first flow channel is an acute angle;
each of an outer sidewall and an inner sidewall of the second outlet is curved and is tangent to an outer wall of the first flow channel, wherein a radius of curvature of the outer sidewall is 30 mm to 40 mm, a central angle d is 10 degrees to 30 degrees, a radius of curvature of the inner sidewall is 5 mm to 20 mm, a central angle e is 10 degrees to 30 degrees, and d>e; and
a distance between a center of the central angle d and a center of the central angle e measured between perpendicular projection points on the first flow channel is 5 mm to 10 mm; and
a fourth group of flow channels, wherein the fourth group of flow channels comprises a second liquid inlet and an arc-shaped channel perpendicular to a horizontal plane in which the second group of flow channels and the first flow channel are located, the arc-shaped channel uniformly surrounds the first flow channel, and the second liquid inlet is in communication with the arc-shaped channel and the first flow channel.

7. A particle sorting method implemented based on the particle sorting device according to claim 1, wherein the particle sorting method comprises following steps:
A1. in the first flow channel, the sheath fluid envelops the sample and flows forward;
A2. a portion of outer sheath fluid enters the first group of flow channels, and remaining sheath fluid continues to envelop the sample and flows forward;
A3. a portion of the outer sheath fluid enters the second group of flow channels; and
A4. the sheath fluid in the first group of flow channels enters the first flow channel from the first outlet in a reverse direction, wherein
when sorting is required, a volume of the first chamber increases, and the sample in the first flow channel sequentially flows through the second inlet and the first outlet, flows forward while carrying the sheath fluid flowing in from the first outlet, and enters the first chamber; and
when sorting is not required, the volume of the first chamber remains unchanged or decreases, the sheath fluid entering the first flow channel from the first outlet flows in the reverse direction and mixes with the sample, and enters the second group of flow channels, and no sample passes through the first outlet.

8. The particle sorting method according to claim 7, wherein the microfluidic chip further comprises:
a third group of flow channels, wherein the third group of flow channels comprises a first liquid inlet and a plurality of channels, the plurality of channels uniformly surround the first flow channel, a second outlet of each channel in the third group of flow channels is in communication with the first flow channel upstream of the first inlet, and a fourth included angle between a liquid flow direction at the second outlet and the liquid flow direction in the first flow channel is an acute angle;
each of an outer sidewall and an inner sidewall of the second outlet is curved and is tangent to an outer wall of the first flow channel, wherein a radius of curvature of the outer sidewall is 30 mm to 40 mm, a central angle d is 10 degrees to 30 degrees, a radius of curvature of the inner sidewall is 5 mm to 20 mm, a central angle e is 10 degrees to 30 degrees, and d>e; and
a distance between a center of the central angle d and a center of the central angle e measured between perpendicular projection points on the first flow channel is 5 mm to 10 mm; and
a fourth group of flow channels, wherein the fourth group of flow channels comprises a second liquid inlet and an arc-shaped channel perpendicular to a horizontal plane in which the second group of flow channels and the first flow channel are located, the arc-shaped channel uniformly surrounds the first flow channel, and the second liquid inlet is in communication with the arc-shaped channel and the first flow channel;
in the step A1, the sheath fluid enters the first flow channel in a following manner:
the sheath fluid entering the first flow channel sequentially through the second liquid inlet and the arc-shaped channel surrounds the sample and envelops the sample; and
thereafter, the sheath fluid enters from the first liquid inlet, then enters the first flow channel from the plurality of channels of the third group of flow channels, envelops the sample, and flows forward.

9. The particle sorting method according to claim 7, wherein the adjustment unit comprises a deformation module and a film, the film separates an interior of the first chamber from an exterior of the first chamber, and the deformation module is arranged on an outer side of the first chamber and connected to the film; and
the volume of the first chamber is adjusted in a following manner:
the deformation module pushes the film in the first chamber to move back and forth, and the volume of the first chamber switches between increasing and decreasing.
